(19) United States
(12) Patent Application Publication
(10) Pub. No.: US 2019/0216207 A1
(43) Pub. Date: Jul. 18, 2019

(54) Skin Treatment Device

(71) Applicant: Telebrands Corp., Fairfield, NJ (US)

(72) Inventors: Lifeng FAN, Yuyao (CN); Junchao ZHOU, Yuyao (CN)

(21) Appl. No.: 16/210,603

(22) Filed: Dec. 5, 2018

(30) Foreign Application Priority Data
Dec. 8, 2017 (CN) 201721699680.6

(51) Int. Cl. A45D 44/00; A61B 17/54; A61B 17/32; A61H 7/00

(52) U.S. Cl. CPC A45D 44/00; A61B 17/54; A61B 2017/00734; A61H 7/005; A61B 17/32

(57) ABSTRACT

A skin treatment device comprising a skin polishing head to remove skin from a treatment area, a collection bin disposed within the housing and positioned to collect the skin removed by skin polishing head, a conduit disposed adjacent to the skin polishing head and the housing, the conduit being in fluid communication with the collection bin, and a drive mechanism coupled to the skin polishing head, rotation of the drive mechanism simultaneously driving the skin polishing head and creating suction to urge skin removed by the skin polishing head through the conduit and into the collection bin.

### CROSS REFERENCE TO RELATED APPLICATIONS

This application claims the benefit of Chinese Patent Application No. 201721699680.6, filed Dec. 8, 2017, which is hereby incorporated by reference in its entirety.

### FIELD

Embodiments of the present invention relate generally to a skin treatment device. More specifically, embodiments of the present invention relate to a motorized skin treatment device having a vacuum for removing and collecting removed skin.

### BACKGROUND

A callus is a thickened and/or hardened part of the skin and can often be found on hands and feet. Often, the callus is dead skin. Calluses can appear on feet due to rubbing from poorly fitting shoes or excessive exercise, and can commonly be found on the heel, sides of the toes, or the ball of the foot. Calluses can appear on hands from rubbing and pinching of skin that occurs while lifting heavy objects or from regular weightlifting and can commonly be found on the palms right below the fingers.

There are a variety of known methods of removing calluses, most of these methods involve scraping, shaving, or rubbing the calluses until the dead skin has been removed. Other methods include softening the callus by soaking the callus in warm water or using lotions or creams. Known devices used to remove calluses or dead skin, include pumice stones, foot files, and a variety of abrasive devices. Some of these devices are electrically powered and include a rotating or vibrating abrasive head.

The known abrasive skin treatment devices have certain shortcomings. For example, dislodged dead skin can adhere to the mechanical structure, such as in the drive shaft, gears, etc. of the abrasive devices, accumulate on objects located in the vicinity of the user, or even remain on a user's body. Accordingly, the device may need to be dismantled and cleaned, or the user's body may need to be cleaned. Additionally, known abrasive skin treatment devices can be expensive to purchase and use, have a short duration of use due to limited battery life, and poor reliability and poor vacuum suction due to mechanical limitations.

### SUMMARY

Embodiments of the present invention can provide an exemplary skin treatment device. The exemplary skin treatment device can include a skin polishing head to remove skin from a treatment area, a collection bin disposed within the housing and positioned to collect the skin removed by skin polishing head, a conduit disposed adjacent to the skin polishing head and the housing, the conduit being in fluid communication with the collection bin, and a drive mechanism coupled to the skin polishing head, rotation of the drive mechanism simultaneously driving the skin polishing head and creating suction to urge skin removed by the skin polishing head through the conduit and into the collection bin.

According to certain exemplary embodiments, the drive mechanism can include a drive motor, a drive power supply, a switch, and a drive shaft. According to certain exemplary embodiments, the drive motor can be a double-headed motor. According to certain exemplary embodiments, the movement imparted by the drive mechanism can be rotational movement about a longitudinal axis of the skin treatment device. According to certain exemplary embodiments, the skin treatment device can further include at least one gear operatively coupled to the drive mechanism and the skin polishing head to enable the drive mechanism and the skin polishing head to rotate at different speeds.

According to certain exemplary embodiments, the skin polishing head can include a support shaft and a polishing piece, wherein the support shaft can be operatively coupled to the drive mechanism and the polishing piece can be removably coupled to the support shaft. According to certain exemplary embodiments, the skin treatment device can further include a polishing protective cover removably coupled to skin polishing head.

According to certain exemplary embodiments, the skin treatment device can further include an impeller, the impeller being driven by the drive mechanism to rotate the impeller to thereby create an air flow which creates the suction.

Another embodiment of the present invention can provide another exemplary skin treatment device. The skin treatment device can include a rotating skin polishing head to remove skin from a treatment area and a rotating impeller which creates a suction to draw the skin removed by the polishing head from the treatment area.

According to certain exemplary embodiments, the skin treatment device can further include a drive mechanism coupled to the skin polishing head and impeller, rotation of the drive mechanism simultaneously driving head and the impeller. According to certain exemplary embodiments, the skin treatment device can further include at least one gear operatively coupled to the head and the impeller to enable the head and impeller to rotate at different speeds.

According to certain exemplary embodiments, the skin treatment device can further include a conduit adjacent to the polishing head and in fluid communication with the impeller such that the suction created by the impeller urges skin removed by the polishing head through the conduit.

According to certain exemplary embodiments, the head can be interchangeable. According to certain exemplary embodiments, the interchangeable head can include at least one of an abrasive head and a massage head. According to embodiments of the present invention, the interchangeable head can have a concave surface.

According the certain exemplary embodiments, the skin treatment device can further include a collection bin.

According to certain exemplary embodiments, the housing of the skin treatment device can be ergonomically shaped. According to certain exemplary embodiments, the skin treatment device can further include a light source configured to indicate an operational mode of the skin treatment device. According to certain exemplary embodiments, the power supply can include a rechargeable power source.

### BRIEF DESCRIPTION OF THE DRAWINGS

The features and advantages of the present invention can be more readily understood from the following detailed description with reference to the accompanying drawings, wherein:

FIG. 1 is a side perspective view of an exemplary skin treatment device according to an embodiment of the present invention;

FIG. 2 is an exploded view of an exemplary skin treatment device according to an embodiment of the present invention;

FIG. 3 is a cross-sectional view of an exemplary skin treatment device according to an embodiment of the present invention;

FIG. 4 is a close-up view of an exemplary head portion according to an embodiment of the present invention;

FIG. 5 is a side perspective view of an exemplary skin treatment device according to an embodiment of the present invention;

FIG. 6 is an exploded view of an exemplary skin treatment device according to an embodiment of the present invention;

FIG. 7 is a cross-sectional view of an exemplary skin treatment device according to an embodiment of the present invention;

FIG. 8 is an exploded view of an exemplary head portion according to an embodiment of the present invention;

FIG. 9 is a perspective view of an exemplary head portion according to an embodiment of the present invention;

FIG. 10 is an exploded view of an exemplary head portion according to an embodiment of the present invention; and

FIG. 11 is a cross-sectional view of an exemplary head portion according to an embodiment of the present invention.

### DETAILED DESCRIPTION

Embodiments of the present invention generally relate to a skin treatment device. More specifically, embodiments of the present invention relate to a motorized skin treatment device for removal of calluses and dead skin and can include a vacuum and a collection bin for collecting and storing the removed skin.

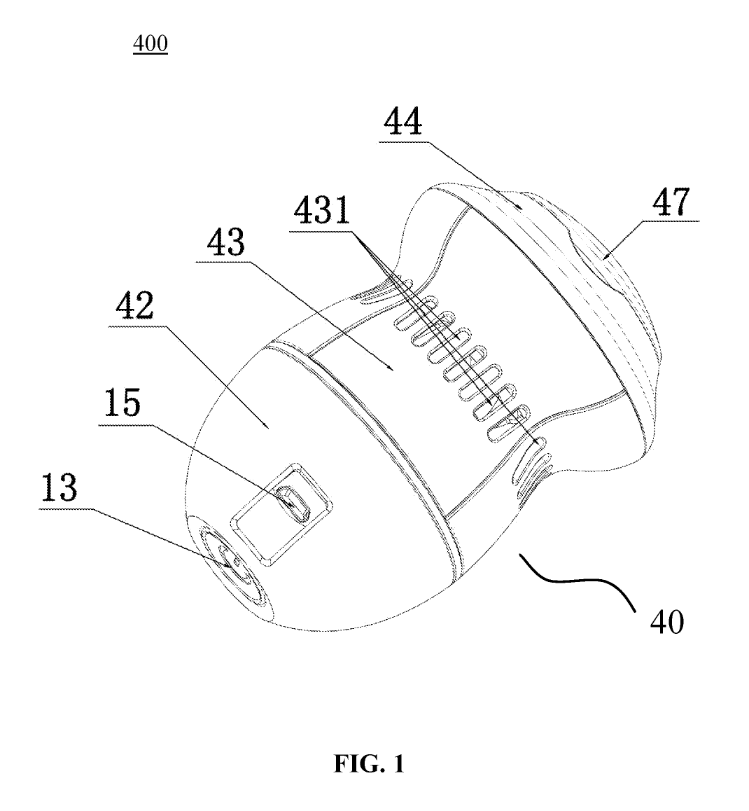

FIGS. 1-3 show an exemplary skin treatment device 400 according to an exemplary embodiment of the present invention. Skin treatment device 400 can include a housing 40, drive mechanism 30, skin polisher 47 and a centrifugal impeller 31. Drive mechanism 30 can be operatively coupled to skin polisher 47 and centrifugal impeller 31 thereby allowing synchronized operation of the skin polisher 47 and centrifugal impeller 31. Additionally, skin treatment device 400 can include a collection bin 32 to collect skin removed from a treatment area by the skin treatment device 400. In operation, drive mechanism 30 simultaneously drives skin polisher 47 and centrifugal impeller 31. Rotational movement of skin polisher 47 can be used to remove calluses or dead skin from a treatment area. Additionally, rotational movement of centrifugal impeller 31 can create a vacuum to timely suction dead skin and debris from the treatment area into a collection bin 32, where it is filtered and gathered.

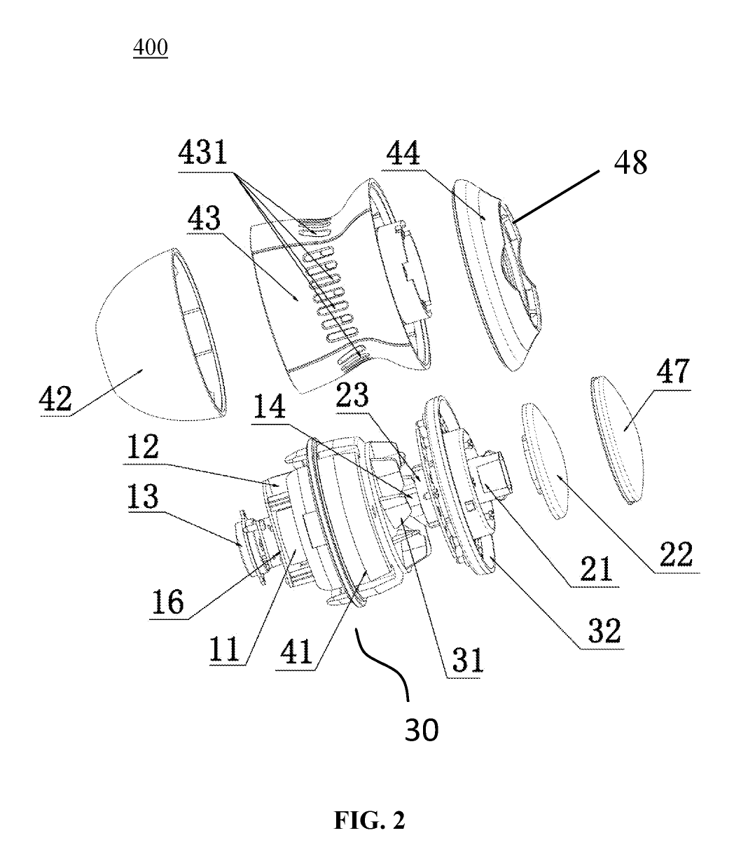

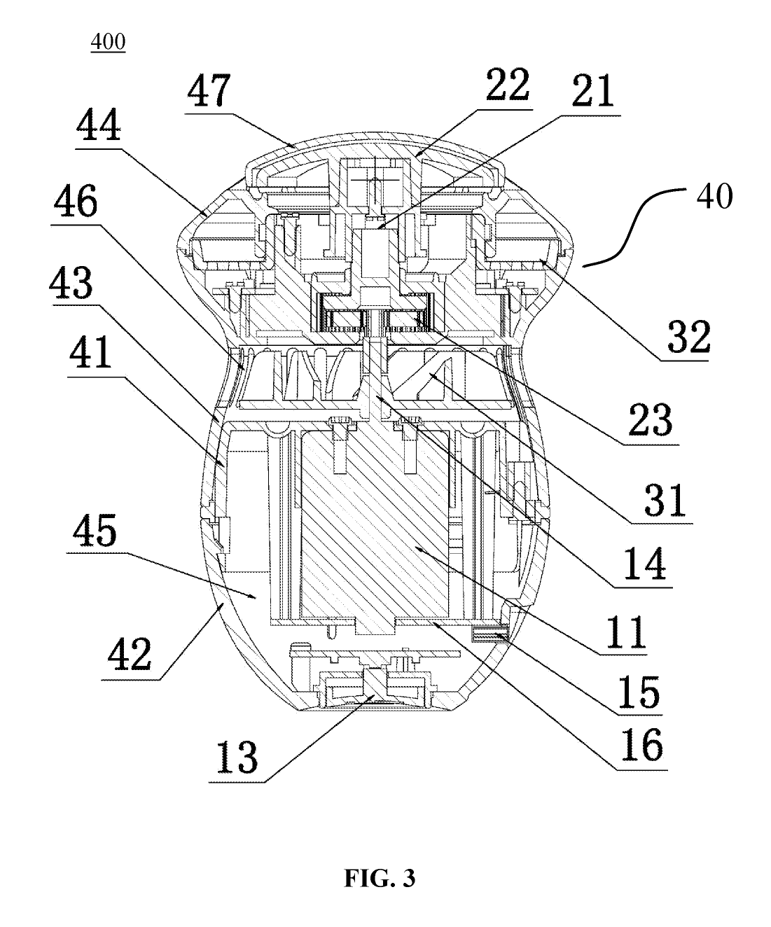

As shown in FIGS. 1-3, housing 40 can include a drive installation pedestal 41, a drive protection housing 42, a centrifugal protection housing 43, and a polishing protection housing 44. As shown in FIGS. 2-3, centrifugal protection housing 43 can be matched and coupled with drive installation pedestal 41 on one end and collection bin 32 on the other to form a centrifugal cavity 46. Additionally, as shown in FIG. 3, drive protection housing 42 can be matched and coupled with drive installation pedestal 41 to form a drive installation cavity 45 designed and configured to house drive mechanism 30. Further, polishing protection housing 44 can be matched and coupled with collection bin 32, thereby coupling polishing protection housing 44 to centrifugal protection housing 43.

As shown in FIGS. 2-3, installation cavity 45 can be designed and dimensioned to house drive mechanism 30. Drive mechanism 30 can include drive motor 11, drive power supply 12, and drive shaft 14. Drive power supply 12 can provide electrical power to drive motor 11 which can provide rotational movement to drive shaft 14. Installation cavity 45 can additionally house a drive power supply 12 configured to supply electrical power to drive motor 11. Drive power supply 12 can be any device configured to store electrical energy, such as a rechargeable battery, thereby allowing the skin treatment device to be portable. As shown in FIGS. 1 and 3, skin treatment device can include button switch 13 located on an external surface of drive protection housing 13 and configured to turn on/off drive power supply 12. As shown in FIG. 1, power supply 12 may be recharged via charge port 15. Charge port 15 can be located on the outside of drive protection housing 13 and can be a micro USB port so that power supply 12 can be charged using any ordinary USB power outlet. Additionally, drive motor 11, drive power supply 12, button switch 13, and charge port 15 can all be electrically connected via a circuit board 16.

Button switch 13 can have a number of modes corresponding to different operational modes of skin treatment device. For example, a long press of button switch 13, such as for 3 seconds, can power on skin treatment device. Similarly, a subsequent long press of button switch 13, such as for 3 seconds, can power off the skin treatment device. Additionally, skin treatment device can have more than one operational speed. For example, skin treatment device can have two operational speeds: slow and fast. In one embodiment, skin treatment device can be configured to operate on slow operational speed when it is first turned on. A subsequent short press of button switch 13 can change the operational speed to high and a third short press of button switch 13 can power down skin treatment device. Alternatively, button switch 13 may include a three way switch having three positions, such as, for example, a rocker switch, a toggle switch, etc. and may be articulated between various positions.

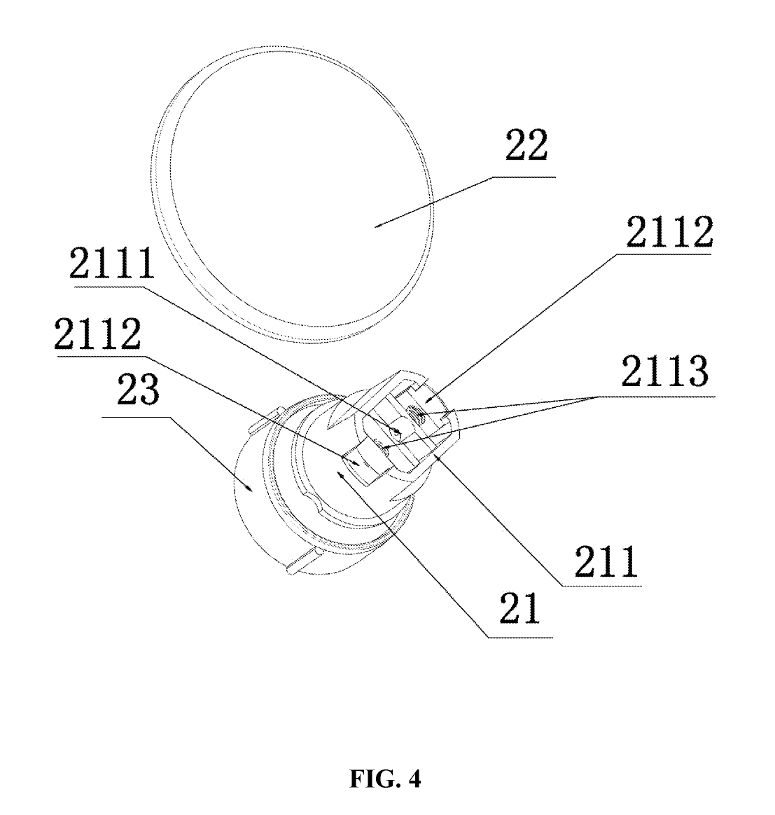

As shown in FIGS. 2-3, drive shaft 14 can extend from installation cavity 45 into centrifugal cavity 46 where it can be operatively coupled to a centrifugal impeller 31. Additionally, as shown in FIGS. 1-3, centrifugal impeller 31 can be housed within centrifugal cavity 46 and can be aligned with vent holes 431 on centrifugal protection housing 43. As shown in FIGS. 2-3, drive shaft 14 can additionally be operatively coupled to support shaft 21. For example, as shown in FIGS. 3-4, skin treatment device 400 can include planetary gear 23 configured to operatively couple support shaft 21 to drive shaft 14. Preferably, planetary gear 23 can be designed and configured to reduce the rotation speed transmitted from drive shaft 14 to support shaft 21.

As shown in FIGS. 2-4, support shaft 21 can be configured to releasably couple to a number of attachments, such as a polishing piece 22. For example, support shaft 21 can include a dismountable snap-fit connection to releasably couple to polishing piece 22. Polishing piece 22 can be mushroom-shaped and include an abrasive surface for exfoliation of the treatment surface. Polishing piece 22 can be substituted by other polishing heads that enable trimming and polishing of dead skin and callouses. For example, a variety of polishing heads can be used to trim and polish the foot sole callus, foot pad, etc, or to resolve problems such as an embedded nail, heavy nail and/or ringworm of the nails, etc.

As shown in FIGS. 1-3, the skin treatment device can include polishing protection cover 47 and polishing piece 22. As shown in FIG. 4, polishing piece 22 can be coupled to support shaft 21 using snap-fit structure 211. Preferably, snap-fit structure 211 is of non-circular form. Snap-fit structure 211 can include positioning socket connection hole 2111, two reset fixture blocks 2112, and reset springs 2113. Fixture blocks 2112 can be positioned symmetrically across each other on opposite sides of snap-fit structure 211 via reset springs 2113. As shown in FIG. 4, shaft lever of polishing piece 22 can be matched and coupled to snap-fit structure 211. The middle position of the socket connection hole 2111 can be arranged with positioning shaft. The walls of the two sides of socket connection hole 2111 are arranged with slot which is matched with two reset fixture blocks 2112. Polishing piece 22 and support shaft 21 can be matched and socket connected to snap-fit structure 211 via shaft lever. Additionally, positioning shaft and positioning socket connection hole 2111 can be matched and positioned and reset fixture block 2112 and slot are matched and snap fitted to realize fixing and snap-fit connection. This embodiment has a simple structure and can be renovated conveniently.

As shown in FIG. 3, polishing piece 22 is exposed on the outside of polishing protection housing 44. Skin treatment device can include polishing protection cover 47 to protect polishing piece 22 when the device is not being used by a user. Polishing protection cover 47 can be matched and socket connected with polishing piece 22 using any number of socket connections, such as, snap-fit connection. Additionally, as shown in FIG. 2, polishing protection housing 44 is arranged with holes 48 around its perimeter which run through the collection bin 32 on the of polishing piece 22. As shown in FIGS. 2-3, collection bin 32 can be positioned between centrifugal impeller 31 and polishing piece 22.

In operation, drive motor 11 causes rotation movement of drive shaft 14. Rotational movement of drive shaft 14 is transferred to centrifugal impeller 31 and support shaft 21 via planetary gear 23. Rotation of centrifugal impeller 31 causes airflow in centrifugal cavity 46, and thereby causes air to flow from around the polishing piece 22 through holes 48 into collection bin 32, and out of vent holes 431, thereby creating a vacuum effect. Specifically, the mechanical structure of centrifugal impeller 31 creates negative pressure as a result of high-speed rotation and causes the air intake of to create a suction force resulting from difference in air pressure. As a result of this vacuum, dead skin is timely suctioned into the collection bin 32, where it is filtered and gathered. After use, collection bin 32 can be conveniently and practically dismantled and cleaned. Additionally, rotation of support shaft 21 via planetary gear 23 is transferred to polishing piece 22. This may be helpful, for example, to enable centrifugal impeller 31 and foot skin polisher 22 to be simultaneously driven by the same drive shaft 14. Furthermore, planetary gear 23 reduces the rotational speed of support shaft 21, and therefore of polishing piece 22, relative to drive shaft 14 and centrifugal impeller 31. It may be beneficial to reduce the rotational speed of polishing piece 22 to avoid injuring a user during use of the skin treatment device.

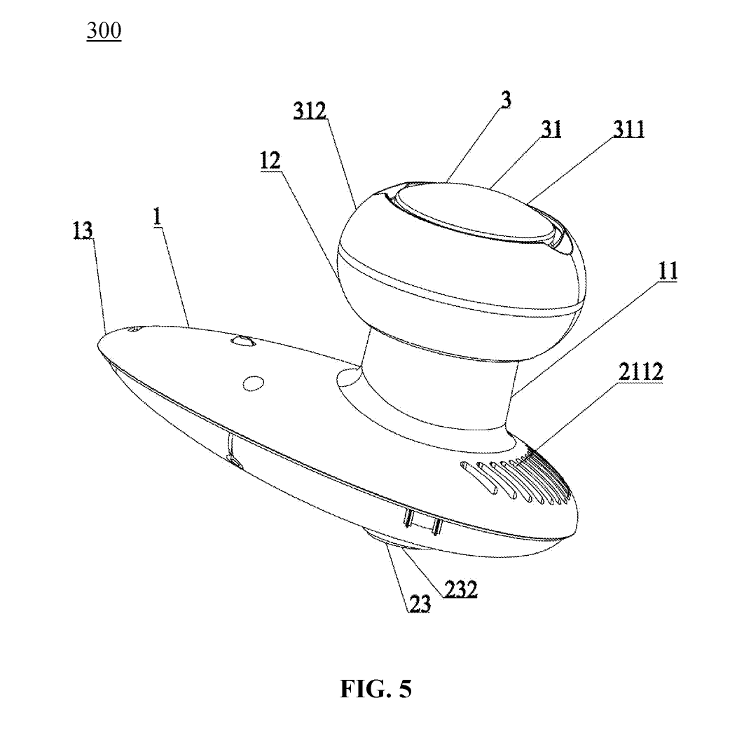

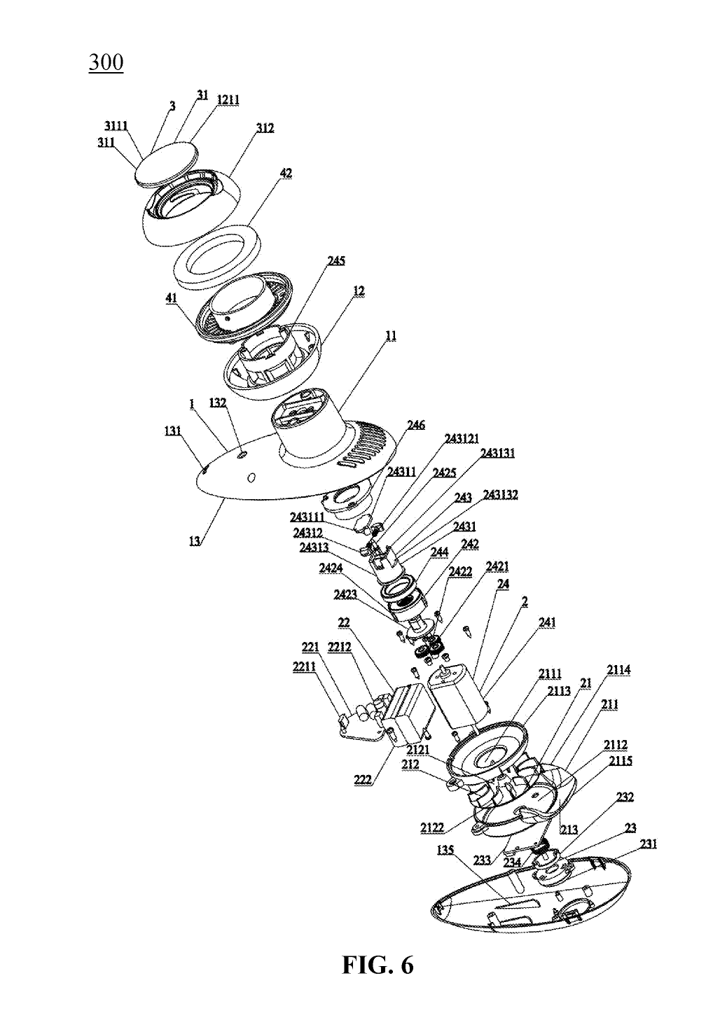

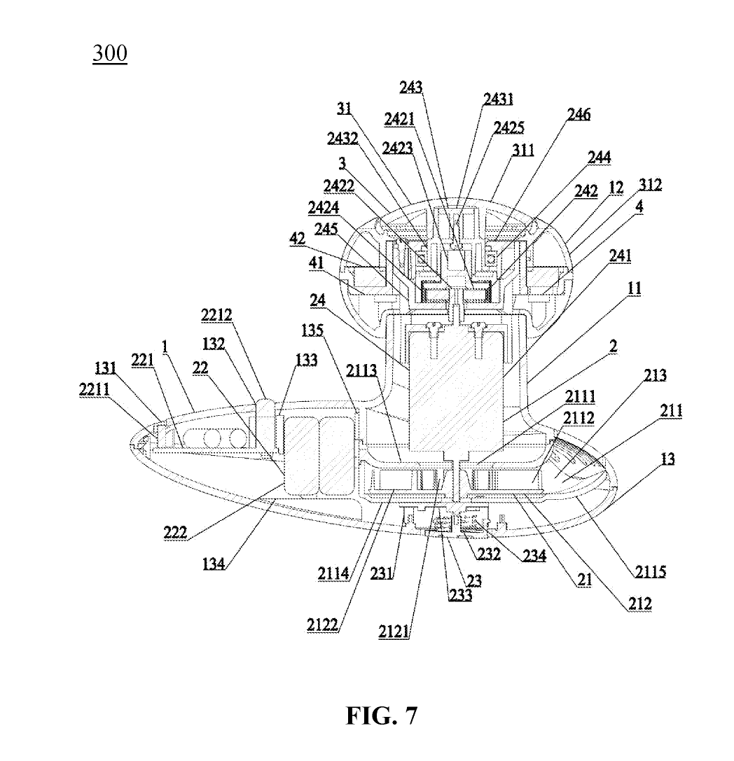

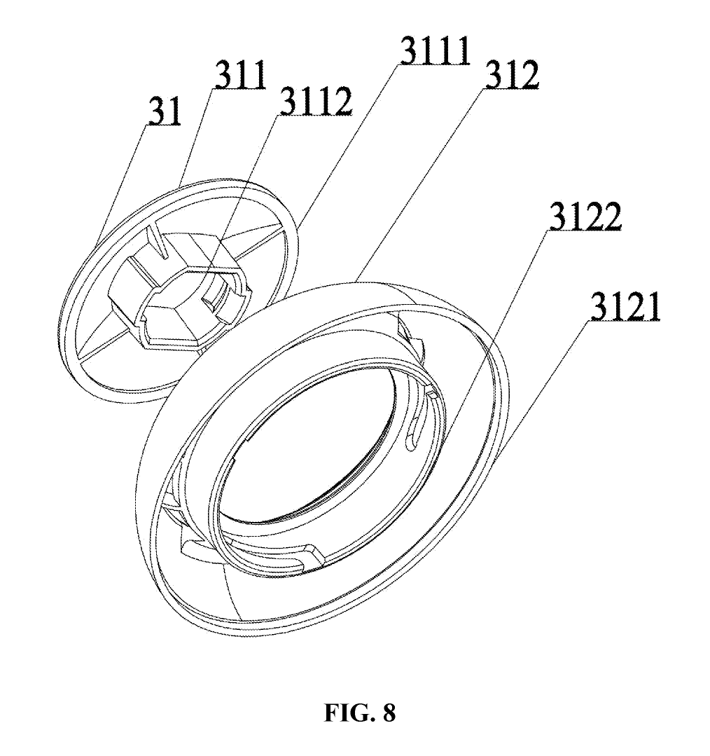

FIGS. 5-11 show a skin treatment device 300 according to an exemplary embodiment of the present invention. As shown in FIGS. 6-7, skin treatment device 300 can include housing 1, main engine 2, replaceable head 3, and collection bin 4. Housing 1 can include cylindrical shell 11, disc-shaped shell 12 and spindle shell 13. Spindle shell 13 can include a large storage cabin with an ergonomic shape to accommodate storage of a greater number of batteries, so that the product has a longer service time. Additionally, spindle shell 13 can include air exhaust device 21, power supply device 22, and control device 23. Cylindrical shell 11 can include a double-head motor 241, and disc-shaped shell 12 can include gearbox 242. Finally, collection bin 4 can be positioned on the top of the disc-shaped shell 12.

As shown in FIGS. 5-7, spindle shell 13 can include first opening 131, second opening 132, first flange 133, second flange 134, and flange 135. The upper surface of spindle shell 13 can be flanged inward to form first flange 133 while lower surface of spindle shell 13 can be flanged inward to form second flange 134. The side wall can be connected to the upper cover plate 2113. Spindle shell 13 can be flanged outward to form third flange 135. First flange 133, second flange 134 and third flange 135 are designed to provide for battery storage and provide an ergonomic shape for ease of use.

As shown in FIGS. 5-7, main engine 2 can be housed within housing 1 and can include an air exhaust device 21. Main engine 2 can further include power supply device 22, control device 23 connected to the power supply device 22, and drive mechanism 24. Power supply device 22 can include first PCB board 221 and storage battery 222. First PCB board 221 can be in electrical connection with storage battery 222, charging socket 2211, and LED lamp 2212. LED lamp 2212 can be configured to indicate various operational modes of skin treatment device 300. Storage battery 222 can be a rechargeable battery, such as, for example, a lithium ion battery, or an alkaline battery. Additionally, charging socket 2211 can be a USB jack commonly used for providing electrical power to a device, and can be configured to enable charging of rechargeable storage battery 222. Charging socket 2211 can face the upper surface of spindle shell 13 and can be matched with first opening 131 on the upper surface of spindle shell 13. LED lamp can face and passes through second opening 132 on the upper surface of spindle shell 13.

As shown in FIGS. 5 and 7, control device 23 can include button switch protection shell 231, button switch 232, second PCB board 233 and extension spring 234. Button switch 232 can be housed within button switch protection shell 231 and can be positioned on an external surface of housing 1. Second PCB board 233 can be in electrical connection with button switch 232 and storage battery 222. Button switch 232 can be used to control the operating status of the skin treatment device. Button switch 232 can be set with a circular extension spring 234 configured to enable button switch 232 to toggle between various operating settings of skin treatment device.

As shown in FIGS. 5-7, air exhaust device 21 can include air exhaust cabin 211. Air exhaust cabin 211 can include upper cover plate 2113 and lower cover plate 2114. An edge of upper cover plate 2113 of air exhaust cabin 211 can be coupled with an edge of lower cover plate 2114 of air exhaust cabin 211. Upper cover plate 2113 can include a sunken edge and lower cover plate 2114 can include a raised edge and air deflector 2115. Air exhaust cabin inlet 2111 can be positioned in the middle of upper cover plate 2113, and lower cover plate 2114 can be positioned with air exhaust cabin outlet 2112. Air exhaust cabin outlet 2112 can be coupled to housing 1 adjacent to several vents through air deflector 2115. Air deflector 2115 can be coupled to the bottom surface of lower cover plate 2114. Air deflector 2115 can form air guiding groove 213 and can have a concave shape corresponding to housing 1. As shown in FIGS. 2 and 3, air deflectors 2115 can be located at one or both ends of air exhaust cabin outlet 2112. The structure of air deflector 2115 can be optimized further so as to effectively enhance the air exhaust effect of air exhaust cabin 211. Air exhaust device 21 can additionally include air guiding groove 213 such that air exhaust cabin outlet 2112 can be position adjacent to air guiding groove 213 thereby enhancing the exhaust of air from air exhaust cabin 211.

Additionally, air exhaust device 21 can include centrifugal impeller 212 which can be housed within the independent air exhaust cabin 211. Centrifugal impeller 212 can include several centrifugal blades 2122, single axial air inlet 2121 and single radial air exhaust. Centrifugal impeller 212 can be housed between upper cover plate 2113 and lower cover plate 2114 of air exhaust cabin 211, and the diameter of air exhaust cabin inlet 2111 can be slightly less than that of centrifugal impeller air inlet 2121. This compact structure can greatly reduce the turbulent flow inside air exhaust cabin 211 so that wind pressure can be effectively formed inside air exhaust cabin 211 and the air exhaust effect can be heightened.

As shown in FIGS. 6-7, drive mechanism 24 can include double-head motor 241, gearbox 242, and a switching device of functional head 243. One end of the double-head motor 241 can be coupled to air exhaust device 21 and the other end can be connected to switching device of functional head 243 to activate switching device functional head 243 via gearbox 242. As a result, double-headed motor 241 enables gearbox 242 transmission mechanism and air exhaust device 21 to rotate at a different speed. Additionally, drive mechanism 24 can include a first rolling bearing 244 positioned on switching device of functional head 243. First rolling bearing 244 stabilizes transmission of rotational movement from gearbox 242 to switching device of functional head 243.

As shown in FIGS. 6 and 7, drive mechanism 24 can include gearbox 242 mounted on gearbox mounting seat 245. Gearbox 242 can be a single-stage or multi-stage gearbox. Gearbox 242 can include planet gear 2421, sun gear 2422, planet carrier 2423, gear ring 2424 and gearbox output shaft 2425. Sun gear 2422 can be set in gear ring 2424 and the planet gear 2421 can be set between sun gear 2422 and gear ring 2424. Planet gear 2421 and sun gear 2422 are external gears and gear ring 2424 is an internal gear. Planet gear 2421 and gear ring 2424 are meshed internally, and planet gear 2421 and sun gear 2422 are meshed externally. The driving shafts of sun gear 2422 can be coaxially fixed with driving shaft of double-head motor 241. Planet carrier 2423 can be matched and coupled to transmission mechanism of gearbox 242 via planet gear 2421. Additionally, gearbox output shaft 2425 can be matched and coupled to transmission mechanism of gearbox 242 via planet carrier 2423 at one end and coaxially fixed with switching device of functional head 243 at the other end. Furthermore, as shown in FIGS. 6 and 7, bearing seat 246 can be set between gearbox mounting seat 245 and switching device of functional head 243. First rolling bearing 244 can be positioned between bearing seat 246 and switching device of functional head 243. The planet gear reducing mechanism described herein provides improved control over the transmission of rotational movement of double-headed motor 241.

As shown in FIGS. 6 and 7, collection bin 4 can include sieve plate 41 and sponge 42. Sieve plate 41 is circular disc in shape and can include several air ventilation holes. The inner side face of sieve plate 41 is convex and can be coupled to gearbox mounting seat 245. The outer side face of sieve plate 41 can be coupled to housing 1. The inner side face and the outer side face of sieve plate 41 can form a grove. Sponge 42 can be set with the groove formed between the inner side face and the outer side face of sieve plate 41. Sponge 42 can be designed to absorb skin dust. Collection bin 4 can additionally include a dust-collection spring, which can be small enough to be measured in nanometers. Dust-collection spring can be position on sieve plate 41 to absorb the impact of skin debris. Additionally, collection bin 4 can be positioned between main engine 2 and the replaceable function head 3. Alternatively, collection bin 4 can be position within replaceable function head 3. Collection bin 4 can include several air vents. The total area of the air vent of skin grinding function head 31, the total area of the air vent of the collection bin 4, the total area of the air vent of air exhaust cabin inlet 2111, and the total area of the air vent of air exhaust cabin outlet 2113 increase sequentially. The sequentially sized air vents form a special air duct which sequentially increases in size. Additionally, the sequentially sized air vents produce greater negative pressure as air moves through the special air duct and enhances the suction effect.

As shown in FIGS. 6-7, replacement head 3 can be coupled to main engine 2. Replacement head 3 can be, for example, an abrasive function head 31, a massage head 321, etc. Abrasive function head 31 is configured to exfoliate skin of the treatment surface. Abrasive function head 31 can include grinding head 311, dust-collection shell 312, and various air vents. Grinding head 311 can include grinding disc 3111 and socketing groove 3112. Grinding disc 3111 can be shaped like an umbrella, i.e., concave downwards. Grinding disc 3111 can include grinding head socketing groove 3112 corresponding to the shape of the functional head switching seat 2431. Grinding head socketing groove 3112 can be internally set with necks matching the reset fixture block 24311 on the two symmetry side faces. Dust-collection shell 312 can be circular disc shaped. The outer ring of dust-collection shell 312 can be flanged downward and can form first connecting face 3121 of dust-collection shell 312. Additionally, first connecting face 3121 can include threads to enable attachment to housing 1. Inner ring of dust-collection shell 312 can be flanged downward and can form a second connecting face 3122, which can include several strip-shaped grooves designed to couple to the lugs on the inner side face of sieve plate 41. The inner ring of dust-collection shell 312 can include several air vents for absorption of the foot skin debris. The dust-collection shell 312 can be coupled to housing 1 and grinding head 311 can be coupled to the switching device of functional head 243. Additionally, the two end faces corresponding to the inner ring of dust-collection shell 312 are set with grooves to facilitate the installation of grinding head 311. Skin treatment device 300 can include a protective shell to protect grinding head 311 when not in use.

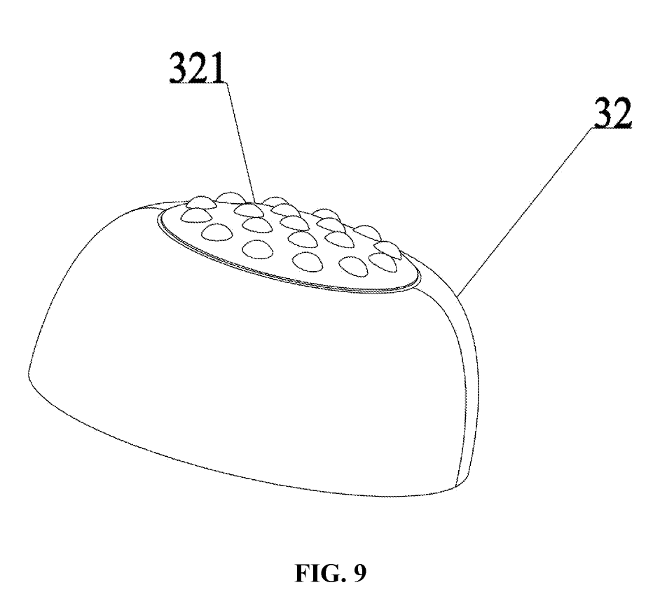

As shown in FIGS. 9-11, skin treatment device can be used for massage by rotating abrasive head 31 and replacing it with a massage function head 321. Massage head 321 can include massage head socketing groove 324, eccentric wheel 322, eccentric shaft 325 and massage head connecting base 326. Massage head 321 is shaped like an umbrella, i.e., concave downward. The edge of massage head 321 can be flanged downward to form a side wall which includes threads and lugs to enable massage head 321 to be coupled to housing 1. Massage head 321 can additionally include several spherical massage noses on the top to perform the massage function. The top of massage head 321 can be flanged inward to form socketing groove 324. Eccentric shaft 325 can be a hollow tube and can include first eccentric shaft 3251 and second eccentric shaft 3252. First eccentric shaft 3251 can be coaxially connected with second eccentric shaft 3252. Additionally, the outer diameter of first eccentric shaft 3251 can be less than that of the second eccentric shaft 3252, and the outer diameter of second eccentric shaft 3252 can be substantially the same as inner diameter of the massage head socketing groove 324. First eccentric shaft 3251 contains massage head socketing groove 324. A second rolling bearing 323 can be positioned between first eccentric shaft 3251 and massage head socketing groove 324. Eccentric wheel 322 can be located inside massage head 321 and second rolling bearing 323 can be located between the massage head 321 and the eccentric wheel 322. Eccentric wheel 322 can be socket connected to second eccentric shaft 3252, and second eccentric shaft 3252 can be coupled to reset fixture block 24311. Eccentric shaft 325 can be coupled to functional head switching seat 2431 via its neck.

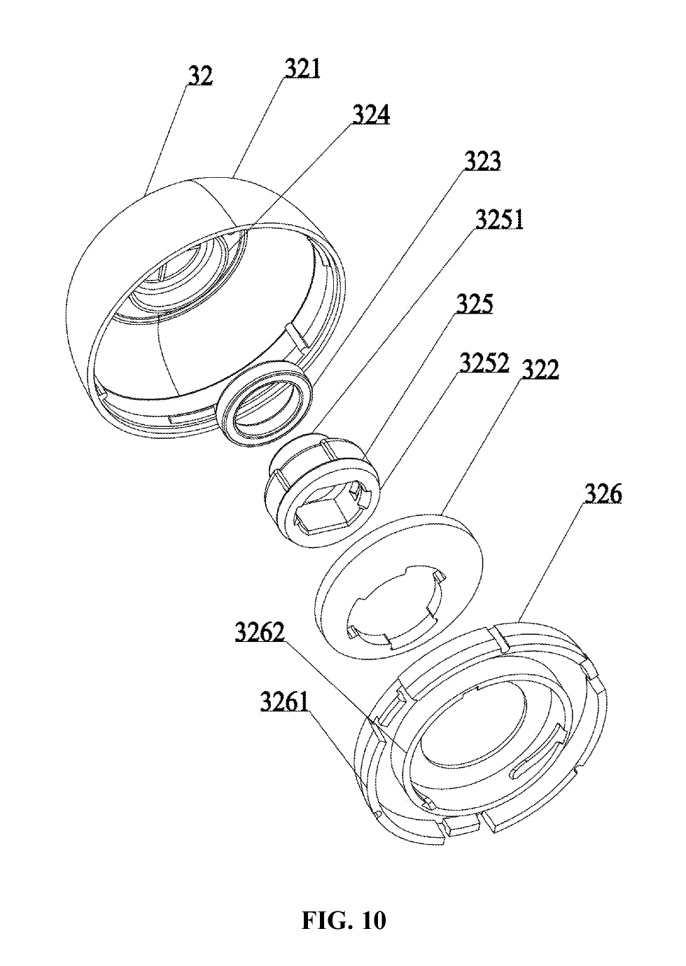

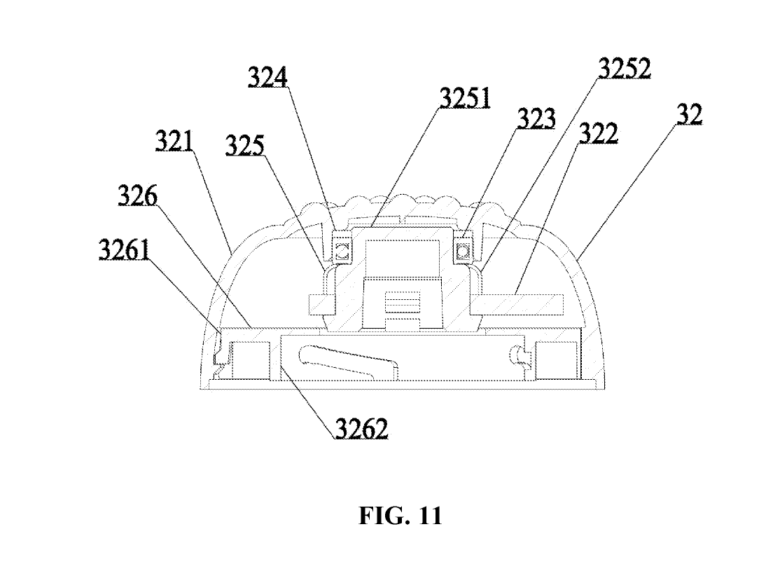

As shown in FIGS. 10-11, massage head connecting base 326 can be circular disc shaped. The inner diameter of massage head connecting seat 326 can be slightly greater than the outer diameter of second eccentric shaft 3252. The two outer rings of massage head connecting base 326 with different diameters flange outward and form first connecting face 3261 and second connecting face 3262. First connecting face 3261 can include several buckles and grooves which are coupled to lugs on the side wall of massage head 321. Second connecting face 3262 of massage head 321 can include several strip-shaped grooves on the inner side face which can be coupled to the lugs on inner side face of sieve plate 41.

As shown in FIGS. 6 and 7, switching device of functional head 243 can include functional head switching seat 2431. Functional head switching seat 2431 can include cover plate 24311, reset fixture block 24312, and supporting seat 24313. Reset fixture block 24312 can allow for quick replacement of replaceable function head 243. Cover plate 24311 can be positioned on top of supporting seat 24313 and can be coupled to the side face of supporting seat 24313 through reset fixture block 24312. Cover plate 24311 can include a centrally positioned hollow socketing bar 243111. The upper part of supporting seat 24313 can be a polygon prism and the lower part can be a cylinder. Reset spring 243121 can be set in reset fixture block 24312. Socketing groove 243131 can be positioned in the center of supporting seat 24313 and can be matched and coupled with socketing bar 243111. Supporting seat 24313 can be positioned using locating necks 243132 on the two symmetrical side faces. First rolling bearing 244 can be positioned at the bottom of functional head switching seat 2431.

The embodiments and examples shown above are illustrative, and many variations can be introduced to them without departing from the spirit of the disclosure or from the scope of the appended claims. For example, elements and/or features of different illustrative and exemplary embodiments herein may be combined with each other and/or substituted with each other within the scope of the disclosure. For a better understanding of the disclosure, reference should be had to the accompanying drawings and descriptive matter in which there is illustrated exemplary embodiments of the present invention.

1. A skin treatment device comprising:
a skin polishing head to remove skin from a treatment area;
a collection bin disposed within the housing and positioned to collect the skin removed by skin polishing head;
a conduit disposed adjacent to the skin polishing head and the housing, the conduit being in fluid communication with the collection bin; and
a drive mechanism coupled to the skin polishing head, rotation of the drive mechanism simultaneously driving the skin polishing head and creating suction to urge skin removed by the skin polishing head through the conduit and into the collection bin.

2. The skin treatment device of claims 1, wherein the drive mechanism includes a drive motor, a drive power supply, a switch, and a drive shaft.

3. The skin treatment device of claim 1, further comprising an impeller, the impeller being driven by the drive mechanism to rotate the impeller to thereby create an air flow which creates the suction.

4. The skin treatment device of claim 1, wherein the skin polishing head includes a support shaft and a polishing piece, wherein the support shaft is operatively coupled to the drive mechanism and the polishing piece is removably coupled to the support shaft.

5. The skin treatment device of claim 1, wherein the movement imparted by the drive mechanism is a rotational movement about a longitudinal axis of the skin treatment device.

6. The skin treatment device of claim 1, further comprising at least one gear operatively coupled to the drive mechanism and the skin polishing head to enable the drive mechanism and the skin polishing head to rotate at different speeds.

7. The skin treatment device of claims 1, further comprising a polishing protective cover removably coupled to skin polishing head.

8. The skin treatment device of claim 1, wherein the housing is ergonomically shaped.

9. A skin treatment device comprising:
a rotating skin polishing head to remove skin from a treatment area; and
a rotating impeller which creates a suction to draw the skin removed by the polishing head from the treatment area.

10. The skin treatment device of claim 9, further comprising a drive mechanism coupled to the skin polishing head and impeller, rotation of the drive mechanism simultaneously driving head and the impeller.

11. The skin treatment device of claims 10, wherein the drive mechanism includes a drive motor, a drive power supply, a switch, and a drive shaft.

12. The skin treatment device of claim 11, wherein the drive motor includes a double-headed motor.

13. The skin treatment device of claim 12, further comprising at least one gear operatively coupled to the head and the impeller to enable the head and impeller to rotate at different speeds.

14. The skin treatment device of claim 9, wherein the head is interchangeable.

15. The skin treatment device of claim 14, wherein the interchangeable head includes at least one of an abrasive head and a massage head.

16. The skin treatment device of claim 15, wherein the interchangeable head has a concave surface.

17. The skin treatment device of claim 9, further comprising a collection bin.

18. The skin treatment device of claim 11, wherein the power supply includes a rechargeable power source.

19. The skin treatment device of claim 11, further comprising a light source configured to indicate an operational mode of the skin treatment device.

20. The skin treatment device of claim 9, further comprising a conduit disposed adjacent to the polishing head and in fluid communication with the impeller such that the suction created by the impeller urges skin removed by the polishing head through the conduit.
